(19) United States
(12) Patent Application Publication
(10) Pub. No.: US 2026/0243648 A1
(43) Pub. Date: Aug. 20, 2026

(54) SYSTEM AND METHOD FOR MEASURING LIQUID PERMEABILITY

(71) Applicant: W.D. Von Gonten Engineering LLC, Houston, TX (US)

(72) Inventors: Ashish Mathur, Houston, TX (US); William D. Von Gonten, JR., Houston, TX (US)

(21) Appl. No.: 19/055,315

(22) Filed: Feb. 17, 2025

(51) Int. Cl. G01N 15/08; G01N 11/00; G01N 24/08; G01N 33/28; G01R 33/50

(52) U.S. Cl. CPC G01N 15/0826; G01N 11/00; G01N 24/081; G01N 33/2823; G01R 33/50

(57) ABSTRACT

A method for measuring steady-state permeability of a rock sample by restoring the permeability samples to their native state of saturation. The method including determining in-situ conditions of the rock sample, including a in-situ saturation, a reservoir temperature, and a reservoir pressure. The rock sample is assessed to determine its water and hydrocarbon saturation and to identify any coring artifacts within the rock sample. The rock sample is then saturated with water and hydrocarbons so that the sample saturation is substantially equal to the in-situ saturation. Once the saturation of the rock sample is verified, reservoir temperature and reservoir pressure are applied to the rock samples and the coring artifacts are monitored. Flow testing is then performed so that a steady-state liquid permeability of the rock sample is measured.

### FIELD OF THE INVENTION

This disclosure relates to a systems and methods for performing steady-state liquid permeability measurements on mudstone rocks.

### BACKGROUND INFORMATION

Conducting steady-state liquid permeability measurements on tight rocks, particularly in mudstone reservoirs with nano-darcy permeability range, presents a set of formidable challenges. The inherent low permeability of these formations demands highly sensitive instruments and techniques to accurately measure the small flow rates. Sample heterogeneity is also a pervasive issue, with mudstone reservoirs samples exhibiting significant layering and heterogeneity in rock fabric and pore structures at various scales. Sample heterogeneity leads to varying flow patterns and flow rates through the sample, discrepancies in results, and requires multiple measurements to define their adequate representative statistical distribution. Microfabric scale effects, including surface roughness, microfractures, and capillary forces, become pronounced in nano-darcy permeability samples, influencing fluid flow behavior, and requiring advanced experimental setups and modeling techniques. Achieving and maintaining representative fluid saturations in reservoir mudstones during experiments is challenging due to the unknown loss of pore fluids during coring and core retrieval. Finally, pressure-dependent permeability behavior in mudstone reservoir samples requires multiple evaluations at different confining and pore pressures and requires analysis of the output data using coupled poro-elastic behavior.

Scale dependency is another challenge in steady-state liquid permeability measurements on nano-Darcy range permeability samples, as macroscopic measurements may not fully capture the effects of nanoscale or microscale features influencing fluid flow. Integrating data across multiple scales is challenging but necessary for a comprehensive understanding. In mudstone reservoirs, interactions between pore fluids and the rock matrix significantly affect permeability, including phenomena such as imbibition, water banking, and near-fracture-face damage. Designing experiments that accurately represent the in-situ conditions of tight rocks requires specialized testing cells, instrumentation, and control of factors such as confining pressure, reservoir pressure, temperature, and pore fluid composition. Finally, data interpretation poses a significant hurdle, with the complex interactions between fluids and rock matrices. Addressing these challenges necessitates a multidisciplinary approach involving geology, engineering, and fluid mechanics, with continuous advancements in experimental techniques, instrumentation, and modeling methodologies crucial for enhancing our understanding of permeability in tight rocks and mudstone reservoirs.

The generation of microcracks and bed partings during coring and core retrieval is a pertinent concern for steady-state liquid permeability measurements, particularly in rocks with nano-darcy range permeability. Coring induced microcracks and bed partings are not present in the undisturbed formation, and their pervasiveness in acquired samples depends on the conditions of coring, the speed of core retrieval, surface core handling and transportation, and these may vary between core sections. If this effect is not removed or minimized during testing it may significantly influence the measurements and provide erroneous and scattered results. Mitigating the impact of microcrack generation during coring and core retrieval involves numerical or analytical modeling of the coring conditions and changes in the core in-situ stress and pore pressure during the process. These result in recommendations on coring procedures and rates of core retrieval that minimize induced damage. Unfortunately, these methods are not fully successful because most of the properties required to conduct the modeling are unknown prior to core testing. Post-coring quality control measures, such as visual inspection and CT scanning of the plugs prepared for testing, are thus commonly conducted to identify and assess the extent of microcrack and larger partings in the retrieved cores. Understanding and quantifying the influence of microcracks on permeability measurements is essential for interpreting laboratory measurements and extrapolating them to reservoir conditions. This is particularly critical in nano-darcy permeability samples where even minor structural changes can have a pronounced effect on fluid flow properties.

Another uncertainty that is introduced by the coring, core retrieval, transportation and overall handling of the core is the loss of native pore fluids (gas, water, and oil), by evaporation. As the in-situ stress is removed, the core expands, the pore pressure is reduced, and water vapor and volatile hydrocarbon fluid components escape the core. The total void volume associated to the combined loss of native pore fluids can be measured accurately on permeability samples. The fraction of the total void volume associated with the evaporated gas, oil and water is unknown but important in obtaining representative permeability measurements.

Lastly, considerations of anisotropy and heterogeneity, which may not be fully captured at the core-scale and sample-scale, present challenges in validating the measurements as well as extrapolating them to the reservoir scale. Mitigating these issues requires evaluating the sample heterogeneity quantitatively, for example using micro-CT image analysis, and imaging the fluid flow during permeability measurements, as it passes through the sample, using high-resolution recurring NMR time-scan measurements, to evaluate the uniformity or non-uniformity of fluid flow through the sample in relation to the homogeneity or inhomogeneity of the sample fabric.

Thus there remains a need for methods for measuring steady-state liquid permeability in laboratory setting that is sufficiently indicative of in-situ conditions to validate the correspondence between rock fabric and fluid flow conditions, or conversely, to understand and quantify the persistence of coring induced artifacts in the measurements.

### FIGURES

FIG. 1 is a flow chart illustrating one embodiment of a method for measuring steady-state permeability.

FIG. 2 illustrates total porosity measured via crush-rock analysis to NMR liquid-filled porosity.

FIG. 3 illustrates as-received pore fluid saturations and post-saturation pore fluid saturations.

FIG. 4 illustrates 2D time lapse T1T2 NMR maps.

FIG. 5 illustrates 3D time lapse scanning during the application of stress cycling to close the existing set of microcracks.

FIG. 6 illustrates pre-and post-test micro-CT imaging and analysis, to evaluate sample quality prior and after testing.

FIG. 7 illustrates 3D NMR time-lapse scan imaging to evaluate fluid flow through fractures, ash-beds, and veins.

FIG. 8 illustrates 3D NMR time-lapse scan imaging to monitor fluid flow patterns within a tight carbonate with a denser inclusion.

FIG. 9 illustrates a 3D NMR time-lapse scan imaging to monitor fluid flow patterns within a tight carbonate with non-homogeneous fabric.

FIG. 10 illustrates the experimental relationship between the logarithm of T2 NMR relaxation and the various field hydrocarbons and reference oil viscosities, which is used to infer the viscosity of any pore-filling hydrocarbon based on NMR T2 measurements.

FIG. 11 illustrates the relationship between fluid temperature and viscosity that is used to correct inferred viscosity values from NMR T2 measurements to their appropriate temperature conditions.

### DESCRIPTION

This disclosure describes new and improved methods for conducting steady-state, liquid permeability measurements on mudstone reservoir samples that allow for minimizing the effect of coring induced artifacts, restoring pore fluid saturation to native conditions, and continuous monitoring of the fluid flow through the sample. The continuous monitoring during permeability testing allows for visualization of the uniform or non-uniform fluid flow through the sample, which by comparison to the homogeneous or heterogeneous rock fabric it can be ascertained if the observed flow profiles is related to the rock fabric, resulting in acceptable measurements, or to induced sample damage, resulting in non-acceptable measurements. In addition, the disclosed methods can evaluate the rheologic properties of native hydrocarbons saturating the rock matrix, at in-situ conditions, based on a comparison with previously obtained collections of data.

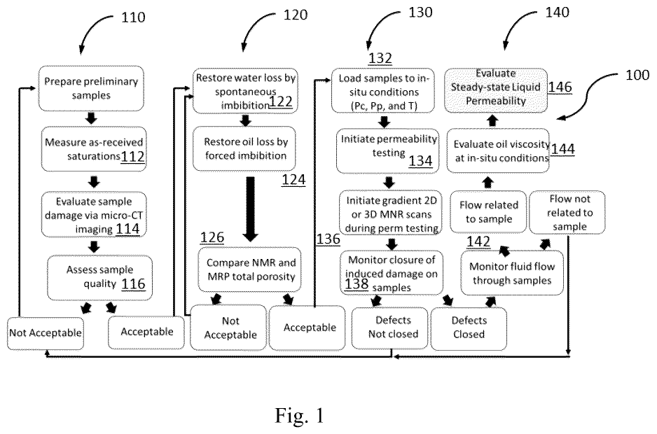

The following disclosure summarizes the preferred methods used for improved steady-state liquid permeability measurements on mudstone rocks, that remove coring induced artifacts, restore fluid saturations to native conditions, and monitor flow homogeneity through the samples. FIG. 1 shows one embodiment of these methods in graphical form. In general, steady-state liquid permeability measurement 100 includes a sample assessment step 110, a sample saturation step 120, a verification testing step 130, and a flow testing step 140.

In a sample assessment step 110, reservoir mudstone samples undergo high-frequency 12 MHz NMR scans (T2, T1T2 scans) 112 in their as-received state of saturation to evaluate fluid saturations, including both water and oil. Micron-resolution micro-CT scanning 114 is then employed to identify microcracks and assess sample heterogeneity based on density contrast. Next, a preliminary sample selection 116 is conducted by applying a predetermined sample rejection criterion that specifies the tolerable and non-acceptable conditions of microcracking and partings that could be accepted for testing. Rejected samples are replaced with substitute samples from similar locations and accepted samples are moved onto sample saturation step 120.

In a sample saturation step 120, accepted samples are first placed in a humidifier 122 to restore lost water due to evaporation during coring, core retrieval, handling, and storage. Once the sample weights stabilize in the humidifier, NMR scanning is repeated to quantify the increase in water content and verify water saturation.

Once humidified, the samples are then saturated with the designated hydrocarbon 124 (formation crude oil or a reference hydrocarbon such as decane) through vacuum saturation followed by pressure saturation in a testing cell subjected to reservoir pressure and temperature conditions. This process can take about a week.

Subsequently, NMR scans and analysis 126 are used to validate that the samples are completely saturated with both water and the desired hydrocabon. For this, the sample NMR liquid porosity may be compared with total porosity measured on a representative twin sample, often taken from the same location as the permeability sample, using crush rock mud rock property analysis (MRP).

Samples showing acceptable NMR and MRP total porosities are selected for further testing. Samples not showing acceptable NMR and MRP total porosities are replaced with substitute samples from similar locations and moved through the same process as the previous. Generally, most samples show similar NRM liquid and MRP total porosity within an error of ±1 p.u.

In a verification testing step 130, the saturated samples are then loaded 132 into a high-pressure, high-temperature core-flooding cell that is invisible to NMR signals. An exemplary testing apparatus is shown and described in U.S. Pat. No. 11,573,191, which is incorporated by reference herein for all purposes. The cell is flooded with a confining fluid (fluorinert) that is also invisible to NMR signals. The samples are brought to their in-situ temperature by heating the confining fluid with a heat tape around the confining fluid tubing vessel and connected to a heat control unit and thermocouple. The hot confining fluid is circulated at a high pump rate (generally 25 cc/min) to facilitate the heat transfer and equilibration. A backpressure regulator connected to the confining tubing maintains the confining pressure on the sample. Inline thermocouples at the top of the sample measure the sample temperature.

The desired injection fluid is applied to the samples to initiate permeability testing 134, while both confining and pore pressures gradually increased in 500 to 1000 psi intervals until reaching the target reservoir pressures. The maximum confining pressure attained may be up to 10,000 psi, with a pore pressure of up to 9,000 psi, and a testing temperature up to 100° C.

Spatial 2D Image and 3D Image NMR scans are then conducted 136 to evaluate the effectiveness of the removal of sample induced damage (microcracks and parted interfaces) by the application of loading and unloading cycles of the confining stress and monitoring the closure of induced sample damage 138. Samples showing adequate closure of induced sample damage continue the process to the subsequent stage of measurements. Samples not showing adequate closure of induced sample damage are replaced with substitute samples from similar locations and moved through the same process as the previous.

After the target confining and reservoir pressures are reached, the sample steady-state permeability is measured in a flow testing step 140 by imposing a differential pressure across the upstream and downstream ends of the sample. The upstream of the sample is connected to the fluid injection pump and the downstream to a back pressure regulator. The upstream pressure is maintained at least 200 psi higher than the downstream pressure. Pump flowrates are monitored and adjusted till a stable differential pressure is achieved.

During steady-state permeability measurements, repeated NMR T1 T2 and T1T2 scans are performed throughout the test, to observe and evaluate changes in sample saturation 142. Spatial, 2D Image and 3D Image NMR scans, are conducted to evaluate the type of fluid flow through the sample (e.g., uniform to non-uniform) and to validate if this is related to the rock fabric (e.g., homogeneous to heterogeneous) or to induced sample damage.

Samples showing adequate relationship between sample fabric and flow type through the sample are selected for data analysis and evaluation of permeability. Samples not showing adequate relationship between sample fabric and flow type are replaced with substitute samples from similar locations and moved through the same process as the previous.

NMR T2 measurement of the bulk oil to viscosity relationships are then used to evaluate the viscosity of the pore-filling oil under the stablished conditions of in-situ pressure and temperature 144. Steady-state liquid permeability is then evaluated using Darcy's Law 146.

After completing the permeability measurements, the samples are removed from the cell, and a subsequent NMR scan may be performed to evaluate post-permeability fluid saturations. Furthermore, post-permeability micro-CT imaging and analysis may also be conducted to evaluate potential alterations in rock fabric, including crack closure, in relation to the corresponding pre-test sample images.

The sample assessment step 110 allows for the reduction or minimization of coring induced artifacts in the sample. As described above, permeability measurements may be strongly affected by presence of micro-cracks, parted laminations, opened fractures and other induced features during coring, core retrieval, and core handling. These features are not present under in-situ conditions and can impact the accuracy of laboratory measurements.

The coring induced artifacts can be reduced, or minimized, by methods that include conducting an as-received, micron-resolution, micro-CT scanning and imaging analysis to evaluate the presence and distribution of artifacts in a sample and apply acceptance/rejection criteria based on the results of the image analysis. Such criteria may include coring induced artifact's concentration, size, and distribution. Although some level of coring induced artifacts may be present, these methods allow for unsatisfactory samples to be identified and rejected or replaced with better quality substitutes.

Sample saturation step 120 seeks to restore the samples back to their native state of saturation (e.g. as present in the formation) prior to testing. Prepared reservoir mudstone samples for steady-state permeability measurements undergo high-frequency 12 MHz NMR scans (T2, T1T2 scans) in their as-received state of saturation, to evaluate fluid saturations, including both water and oil. These samples have a water wet pore system (e.g., clay pores) and an oil-wet pore system (e.g., organic pores). Thus, to restore their native state of saturation the samples are exposed to water wet (water) and oil wet (hydrocarbon) fluids in two consecutive steps and allowed to recover their in-situ state of saturation based on their intrinsic surface energy and capillary forces.

In a water saturation step, the samples may be placed in humidifier or imbibed in formation brine at ambient pressure and temperature to allow them to regain their lost water based on spontaneous imbibition until a thermodynamic equilibration is reached.

In an oil saturation step, which occurs after the water saturation step, the samples may first be vacuum saturated with their native hydrocarbon (i.e., produced oil) and, after equilibration, subjected to pressure saturation under in-situ conditions of reservoir pressure and temperature.

After completing the water and oil saturation steps, the samples may undergo high-frequency 12 MHz NMR scans (T2, T1T2 scans) once again, to measure their fluid-filled (water and oil) porosity. These measured NMR fluid porosity values can be compared to an independent crushed rock measurement of total porosity and if the porosity values acceptably correlate, the samples are considered saturated and are accepted for further testing. Alternatively, the samples can be replaced with substitute samples from similar depth locations or rock types and the evaluation and saturation process performed again.

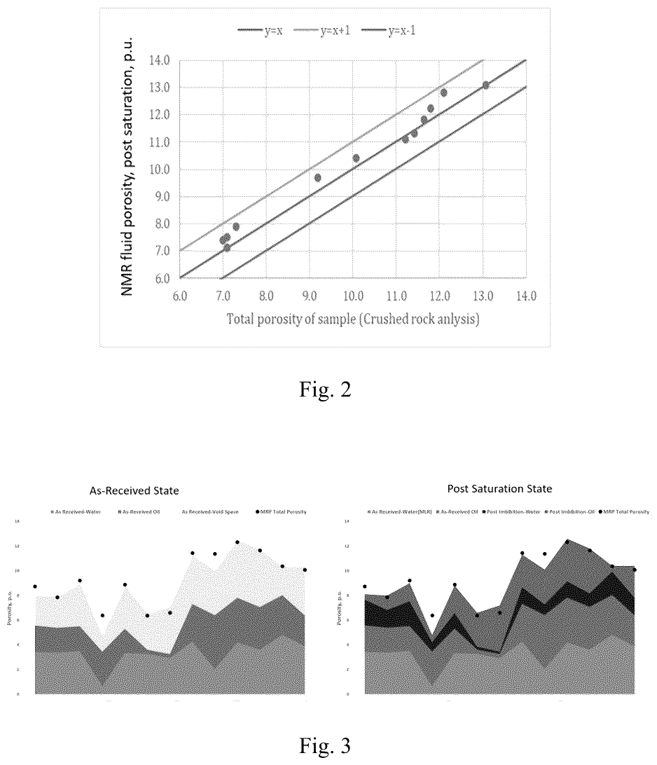

FIG. 2 shows an example of a comparison between total porosity measurements via crushed rock analysis and NMR liquid-filled porosity. A 1:1 line is provided as a reference of identical measurements. Parallel lines above and below this are provided to show a confidence range of ±1 p.u. In this case, the similarity in total porosity between the two methods is shown to be within the specified uncertainty range. All these samples are accepted for further testing. FIG. 3 shows the partition of the originally measured void volume (left) into the corresponding volume fractions of water and oil (right). The black circles in both figures are measurements of crushed rock total porosity and are identical to each other in both plots. Thus, these methods may also allow improvement of the crushed rock measurements of saturation (MRP) by providing a quantitative evaluation of the water and oil lost during coring and core retrieval.

In verification testing step 130, after restoring fluids to their native state, permeability measurements of the samples are acquired using NMR time-lapse scanning procedures. These measurements identify the fluid saturations associated with the microcracks present in the sample and quantify the effect of confining pressure on them, as they close gradually, over time.

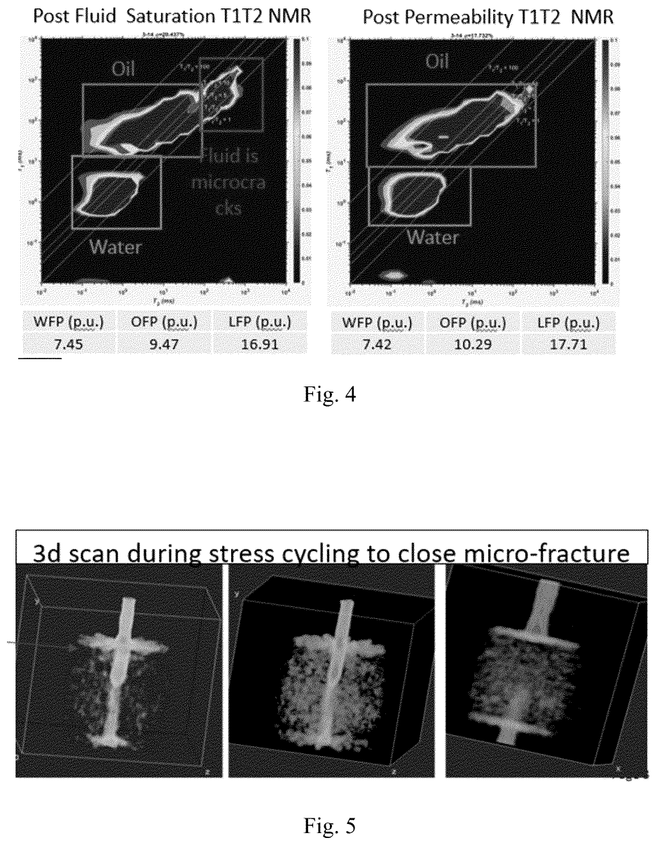

FIG. 4 shows an example of time lapse NMRT1T2 scans showing the initial and final states of a sample with some level of induced microcracking. The figure to the right shows the final state where the increasing confining stress caused the microcrack population to close as this was verified by the elimination of the NMR fluid-filled microcrack signature. Once this occurrence is validated, the permeability measurements are initiated with a quantifiable record of the removal of the coring induced artifacts.

FIG. 5 shows the corresponding evaluation of microcrack closure due to stress ageing for a short duration above the reservoir effective pressure followed by stress cycling back to reservoir stress during fluid flow using 3D time lapse NMR scans. The above process is repeated multiple times to ensure that the microcrack has closed. The left image shows channeled flow through some predominant set of microcracks. The middle and right image shows a more uniformly distributed fluid flow as the confining pressure is cycled and the microcracks close as a result.

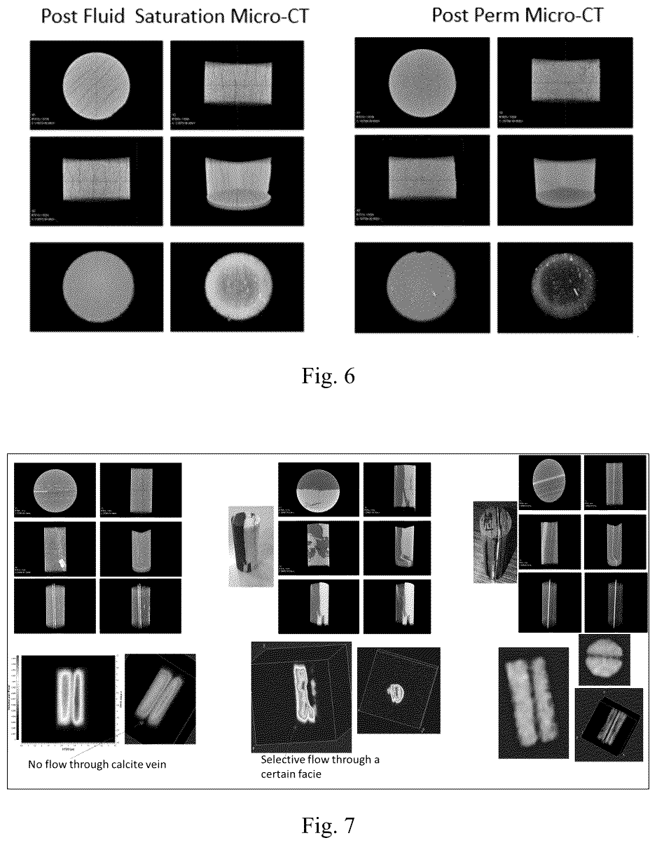

Following the completion of the permeability measurements, post-test micro-CT imaging and analysis can be conducted to verify the absence of open microcracks and other coring induced artifacts, in comparison to what was observed on the corresponding pre-test sample image analysis. FIG. 6 shows an example of a comparison between pre-test micro-CT imaging and analysis(left) and the corresponding post-test micro-CT imaging and analysis(right). The right figure shows the microcracking and bed parting existing in the pre-test sample. The right figure shows the absence of these features after the sample has been subjected to a sufficiently high confining stress and stress cycling.

In flow testing step 140, 2D and 3D time lapse scans are used to evaluate the fluid flow conditions through the samples. In these methods, 3D time laps NMR scans are used to evaluate the fluid flow conditions through the rock matrix and to ascertain if this is influenced by sample fabric, which could be homogeneous or heterogeneous, or the presence of coring induced artifacts. Traditional liquid permeability measurements provide pressure versus flow rate results, as a function of time. However, they do not provide an evaluation of the type of fluid flow the sample experiences (uniform or non-uniform) or the relationship of the rock fabric (homogeneous or heterogeneous) and the fluid flow.

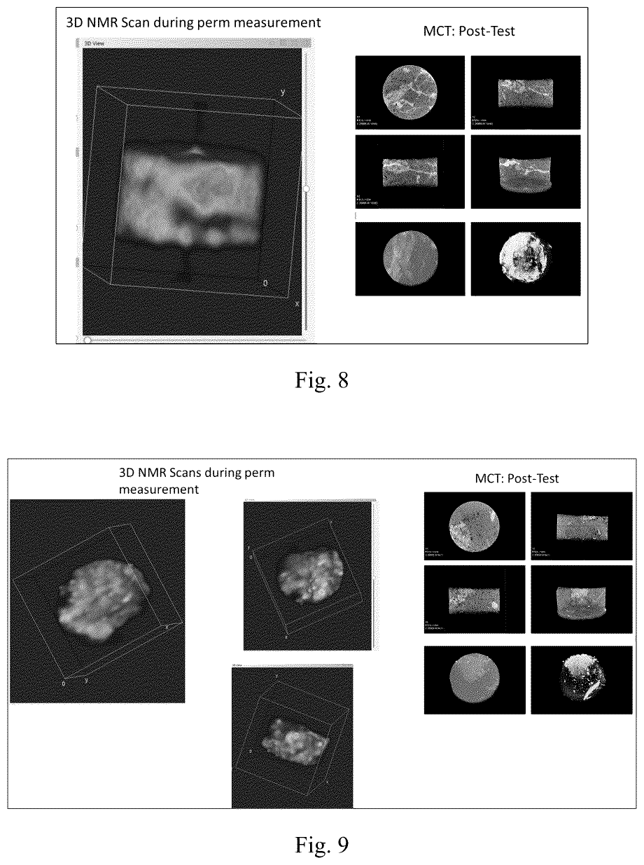

The disclosed methods provide for the assessment of permeability and for propagation of permeability measurements to other locations of the cores or to field logs. FIG. 7 shows examples of these procedures to evaluate fluid flow through fractures (left), ash-beds (middle), and veins (right). FIG. 8 shows an example of the same technique visualizing non-uniform fluid flow resulting from localized sample heterogeneity in a carbonate sample. FIG. 9 shows another example of the same technique visualizing non-uniform fluid flow resulting from distributed sample heterogeneity. If the fluid flow is evaluated to be directly related to the native rock fabric, as in the examples shown, the measurements of pressure gradients and flow rates are accepted for evaluation of permeability. If the fluid flow is evaluated to be unrelated to the native rock fabric and dominated by induced sample damage, the test is concluded and the samples are replaced by substitute samples from similar depths, which are then processed similarly to the others, from the beginning of the workflow.

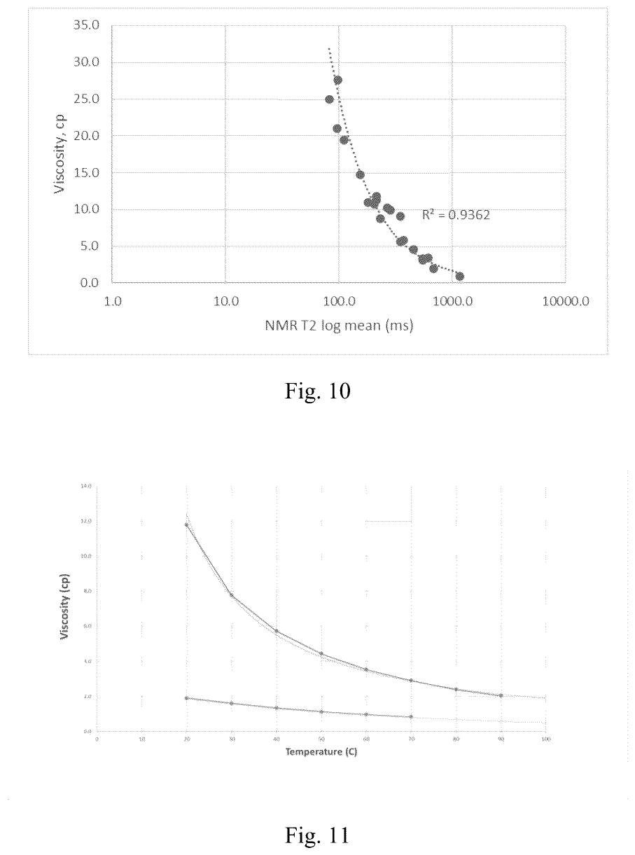

The disclosed methods may also include extrapolating a pore-filling hydrocarbon viscosity as a function of pressure and temperature using NMR measurements using a database of corresponding measurements. The fluid viscosity of a range of field hydrocarbons and reference oils of varying viscosity can be measured using a standard laboratory viscometer at varying temperatures in the range from 15° C. to 100° C. T2 NMR scans for bulk oil samples can also be conducted for these oils and the results used to develop a relationship between the logarithmic mean T2 relaxation time and the oil viscosity (FIG. 10).

FIG. 11 shows the corresponding relationship between viscosity and temperature. The two sets of data allow the evaluation of viscosity for any pore-filling hydrocarbon at the desired condition of in-situ temperature. This procedure speeds up the analysis of permeability measurements considerably. Once the samples and their corresponding measurements of flow and pressure gradients are validated to be representative of the samples tested, the inferred oil viscosity is used for the evaluation of steady-state liquid permeability.

In certain embodiments, the disclosed method may be described as any of the following methods.

A method for conducting improved, higher confidence, steady-state liquid permeability measurements of nano-darcy range permeability tight-rocks and reservoir mudstones.

A method for reducing the uncertainty in steady-state liquid permeability measurements by restoring the permeability samples to their native state of saturation and by validating this condition comparing their liquid-filled NMR porosity to the corresponding crushed rock total porosity.

A method for defining the corresponding fractions of water and liquid hydrocarbons associated to the initially measured “as-received” void volume.

A method for reducing the uncertainty in steady-state liquid permeability measurements by evaluating the coring-induced damage on the samples, selecting the samples with acceptable damage, replacing the samples with unacceptable damage with corresponding better-quality samples, minimizing their effect on the measurements by applying stress cycles, and monitoring the resulting conditions via 2D and 3D time laps NMR scans.

A method for evaluating the flow conditions (uniform to non-uniform) through the sample, via 3D time laps NMR scans and evaluating if these flow conditions are related to the sample fabric (homogeneous to non-homogeneous), or to artifacts associated to coring induced damage.

A method for reducing the uncertainty in steady-state liquid permeability measurements by evaluating the pore-filled hydrocarbon viscosity under various conditions of pressure and temperature, via a combination of NMR T2 scan measurements and database inferred relationships between the two, which were developed over a large set of measurements with the desired range of viscosity

Specific embodiments of the invention are shown by way of examples in the drawings and description. It should be understood, however, that the drawings and detailed description thereto are not intended to limit the claims to the particular form disclosed, but on the contrary, the intention is to cover all modifications, equivalents, and alternatives falling within the scope of the claims.

What is claimed is:

1. A method for measuring steady-state permeability of a rock sample comprising:
determining in-situ conditions of the rock sample, including a in-situ saturation, a reservoir temperature, and a reservoir pressure;
performing a sample assessment step to determine a sample saturation of the rock sample and to identify coring artifacts within the rock sample;
performing a sample saturation step so that the sample saturation is substantially equal to the in-situ saturation;
performing a verification testing step wherein reservoir temperature and reservoir pressure are applied to the rock sample and the coring artifacts are monitored; and
performing a flow testing step wherein a steady-state liquid permeability of the rock sample is measured while applying a differential pressure across the rock sample.

2. The method of claim 1, wherein performing a sample assessment step further comprises:
determining a sample saturation by performing NMR scans of the rock sample; and
identifying coring artifacts by performing micro-CT scans of the rock sample.

3. The method of claim 1, wherein performing a sample saturation step further comprises;
saturating the rock sample with water; and
saturating the rock sample with a hydrocarbon.

4. The method of claim 3, further comprising determining the sample saturation by performing NMR scans of the rock sample.

5. The method of claim 4, further comprising comparing the rock sample to a measured porosity of a crushed rock sample.

6. The method of claim 1, wherein performing a verification step further comprises:
loading the rock sample into a testing cell;
flooding the testing cell with a confining fluid;
heating the testing cell to the formation temperature;
pressurizing the testing cell to the formation temperature; and
acquiring NMR image scans of the rock sample to show coring artifacts.

7. The method of claim 1, wherein performing a flow testing step further comprises acquiring a plurality of NMR image scans to determine flow through the rock sample.

8. The method of claim 7, wherein performing a flow testing step further comprises using NMR T2 measurements to determine viscosity of hydrocarbons in the rock sample.

9. The method of claim 7, wherein performing a flow testing step further comprises using Darcy's Law to determine steady-state permeability.

10. A method for preparing a rock sample for testing comprising:
determining in-situ conditions of the rock sample, including a in-situ saturation, reservoir temperature, and reservoir pressure;
determining a sample saturation of the rock sample;
identifying coring artifacts in the rock sample;
saturating the rock sample so that the sample saturation is substantially equivalent to the in-situ saturation;
disposing the rock sample in a testing cell;
maintaining the testing cell at reservoir temperature and reservoir pressure; and
identifying coring artifacts in the rock sample while the rock sample is in the testing cell at reservoir temperature and reservoir pressure.

11. The method of claim 10, wherein the in-situ saturation and sample suturing both include a water saturation and a hydrocarbon saturation.

12. The method of claim 11, wherein saturating the rock sample further comprises:
saturating the rock sample with water; and
saturating the rock sample with a hydrocarbon.

13. The method of claim 10, wherein sample saturation is measured using NMR scans.

14. The method of claim 10, further comprising comparing the rock sample to a measured porosity of a crushed rock sample.

15. The method of claim 10, wherein coring artifacts are identified using CT scans or NMR scans.
